(19) United States
(12) Patent Application Publication
(10) Pub. No.: US 2025/0387028 A1
(43) Pub. Date: Dec. 25, 2025

(54) AUTO TRIGGER OF MEDICAL IMAGING BY DIAPHRAGM STATE

(71) Applicant: Siemens Healthineers AG, Forchheim (DE)

(72) Inventors: Sunil M K, Bangalore (IN); Salini S, Coimbatore (IN)

(73) Assignee: Siemens Healthineers AG, Forchheim (DE)

(21) Appl. No.: 19/241,899

(22) Filed: Jun. 18, 2025

(30) Foreign Application Priority Data
Jun. 21, 2024 (EP) 24183829.1

(51) Int. Cl. A61B 5/00; A61B 5/28

(52) U.S. Cl. CPC A61B 5/0077; A61B 5/28

(57) ABSTRACT

A system for acquiring a medical image of a patient, comprises: a medical imaging unit configured to acquire the medical image; at least one sensor configured to acquire sensor data of the patient; an input unit configured to receive the sensor data; and a control unit configured to initiate acquisition of the medical image based on the sensor data. The system may automatically initiate acquisition of the medical image based on the diaphragm state of the patient. Accordingly, respective postprocessing algorithms may be rendered unnecessary, and the usage of models and assumptions may be avoided.

### CROSS-REFERENCE TO RELATED APPLICATION(S)

The present application claims priority under 35 U.S.C. § 119 to European Patent Application No. 24183829.1, filed Jun. 21, 2024, the entire contents of which are incorporated herein by reference.

### FIELD

One or more example embodiments of the present invention relate to a system for acquiring medical images for providing an auto trigger of medical imaging by a diaphragm state, a method for using said system and a computer program product.

### BACKGROUND

During a medical examination with the objective to take medical images of a patient, the patient is positioned within and/or in vicinity of a medical imaging unit. Independent of the grammatical term usage, individuals with male, female or other gender identities are included within the term. The medical imaging unit can be, in particular, an x-ray scanner, a mammography scanner, a computed tomography (CT) scanner, and/or the like. Said medical imaging unit is capable of providing an illustration of the organs within the patient, thus enabling enhanced diagnostic mechanisms and/or methods like detecting organ anomalies and/or a possible grow of nodes within the organs. In order to detect anomalies of the organs and/or the growth of nodes within the organs, a number of medical images of the patient are compared with one another, which are taken at different times. In particular, a current medical image is compared with a previous image.

Nonetheless, the organs are situated within the torso of the patient. Thus, the organs of the patient both move and change their geometry depending on a diaphragm state of the patient. The diaphragm state corresponds to a state of the lungs of the patient, in particular whether the lungs are in an inhaled state, an exhaled state, or a state in between. That is due to the fact that the diaphragm, which is the muscle responsible for an inflation and a deflation of the lungs, influences other parts within the torso of the patient as well. Due to internal knowledge of the applicant, the diaphragm state can vary within the number of medical images of the patient which are about to be compared. In consequence, the comparison of the number of medical images may provide a superposition of the influence of a possibly variant diaphragm state and a variation of the organs itself, which may be an indicator for an organ anomaly or a node growth.

According to internal knowledge, said superposition can be addressed by a respective postprocessing of the medical images, wherein a non-rigid registration algorithm and/or a deformable registration algorithm is utilized to artificially manipulate the data within the medical images by “undoing” the effect of the inflation or deflation of the lungs. Nonetheless, the usage of said algorithm is based on a number of models and assumptions. Hence, the degree of uncertainty within the medical image is increased, threatening the precision or even correctness of a diagnosis based on the medical images.

In addition, it is not possible to maintain a specific diaphragm state for all medical images due to physical and/or psychological effects. For example, the patient may experience a panic attack or unconsciousness.

### SUMMARY

It is an object of one or more example embodiments of the present invention to improve the acquiring of medical images.

According to a first aspect, a system for acquiring a medical image of a patient is suggested, the system comprising
a medical imaging unit for acquiring the medical image;
at least one sensor for acquiring sensor data of the patient;
an input unit for receiving the sensor data; and
a control unit for initiating the acquiring of the medical image based on the received sensor data.

The medical imaging unit can be a CT scanner, an x-ray scanner, a mammography scanner, and/or another device configured for taking medical images. The medical imaging unit may comprise a gantry which can have an X-ray source and X-ray detectors which are both rotating within the gantry. The gantry can surround a tube, through which a bed can be moved. The bed can be located on top of a table. The bed can be configured to be movable with respect to an axial direction. The patient can be positioned on the bed in a lying manner.

The system further may further comprise a plurality of sensors. Yet, preferably, the system comprises exactly one sensor for acquiring sensor data of the patient. Hereby, the sensor data may be acquired without any active action of the patient and/or a technician using the system. In particular, pressing a mechanical and/or electronical button or the like may be unrequired in order to do so. In this context, the term “sensor data” means signals or at least one signal from the at least one sensor. The at least one sensor can be configured to acquire data of the patient, representing a physical condition of the patient. The at least one sensor can be attached to the patient and/or be in vicinity of the patient.

The system further comprises the input unit for receiving the sensor data. In particular, the input unit can be configured to convert the sensor data into a computer-readable value or values. The input unit can be configured as a hardware interface for a computer device. In case the system comprises a plurality of sensors, the sensor data of each of the plurality of sensors can be received by the input unit and converted into computer-readable values.

The control unit can be designed using electronical components, in particular, using integrated circuits. Yet, preferably, the control unit can be designed as a computer program product. The control unit can be configured to derive a current diaphragm state of the patient based on the received sensor data. Hence, the control unit can be configured to determine whether at a current time when the sensor data are acquired, the lung of the patient is exhaled, inhaled, or at a state in between. The control unit can further be configured to automatically initiate the acquiring of the medical image based on the determined current diaphragm state. Hence, based on the determined current diaphragm state, the medical imaging unit can be initiated or triggered to automatically capture the medical image. In particular, the acquiring of the medical image can be initiated at the moment in time, when the current diaphragm state of the patient corresponds to a desired diaphragm state. The sensor data and the hereby determined current diaphragm state can thus be used in the manner of an event trigger to initiate the acquiring of the medical image. Alternatively, or additionally, the technician can be informed about the determined current diaphragm state. It is feasible to inform the technician using an acoustical and/or visual indicator.

The diaphragm state corresponds to a state of the lungs of the patient, in particular whether the lungs are in an inhaled state, an exhaled state, or a state in between. That is due to the fact that the diaphragm, which is the muscle responsible for an inflation and a deflation of the lungs, influences other parts within the torso of the patient as well.

In an advantageous manner, the system can automatically initiate the acquiring of the medical image based on the determined current diaphragm state of the patient. That can render respective postprocessing algorithms unnecessary and, thus, can avoid the usage of models and assumptions. Eventually, the diagnosis according to the medical image is both rendered easier and more reliable.

According to an embodiment, the control unit is configured to initiate the acquiring of the medical image in case a current diaphragm state of the patient corresponds to a previous diaphragm state of the patient.

The acquiring of the medical image can be automatically initiated at the moment in time, when the current diaphragm state of the patient corresponds to the previous diaphragm state, wherein the previous diaphragm state corresponds to the diaphragm state of the patient at the time, when a previous medical image has been taken. For example, in case the previous medical image shows the lung in an exhaled state, for the current examination, the system can automatically acquire the current medical image at that moment when the lung is in an exhaled state as well. Hence, the system is configured to continuously acquire sensor data of the at least one sensor and to determine the current diaphragm state until the current diaphragm state and the previous diaphragm state are in agreement with one another. In consequence, the system can automatically enable an equal diaphragm state within all medical images of the patient.

According to a further embodiment, the at least one sensor is an electrode for detecting heartbeats of the patient.

The electrode is configured to detect electrical signals produced by muscle contractions of the patient. In case the patient is not moving, said muscle contractions represent the activity of the heart of the patient. Thus, the electrode is configured to detect the heartbeats of the patient, represented by peaks within the sensor data of the electrode. Therefore, the electrode can be attached to the skin of the patient. Alternatively, or additionally, the at least one sensor can be a camera for capturing a chest of the patient, a respiratory sensor for detecting a breathing condition of the patient, a kinetic sensor for detecting a movement of the patient, a sensor configured to measure a skin temperature and/or a humidity of the skin of the patient, and/or another sensor capable of expressing the physical behavior of the patient. The at least one sensor is compatible with the medical imaging unit. In particular, the at least one sensor is compatible with X-ray radiation and/or magnetic fields. In particular, it is free of interferences with the medical imaging unit. The at least one kinetic sensor can be used to recognize a movement of at least one body part of the patient, for example, a movement of the head, of the chest, or of another body part. The system may comprise a plurality of kinetic sensors, for example two, three, four, or five. The usage of a plurality of kinetic sensors enables the detection of a movement of the patient in a three-dimensional manner. The usage of the at least one sensor enables a precise acquiring of sensor data expressing the physical behavior of the patient.

According to a further embodiment, the control unit is configured to determine the current diaphragm state of the patient based on the detected heartbeats.

The control unit can be configured to analyze and to evaluate the detected heartbeats of the patient. A model can be used to analyze and to evaluate the heartbeats. Furthermore, the control unit can be configured to determine the current diaphragm state based on said analysis and evaluation of the heartbeats. Alternatively, or additionally, the control unit is configured to determine the current diaphragm state of the patient based on the captured movements of the patient. Hereby, photogrammetry can be used in order to determine the diaphragm state of the patient.

According to a further embodiment, the control unit is configured to determine a breathing pattern of the patient based on the detected heartbeats, wherein the control unit is configured to determine the current diaphragm state based on the breathing pattern.

In this context, the term “breathing pattern” represents a breathing behavior of the patient over time. The breathing pattern includes the inhalation and exhalation events of the lung of the patient. For example, characteristics within the breathing pattern can represent an inhalation into the lung of the patient. Hence, the breathing pattern can correspond to the location and/or the movement of the diaphragm. The control unit is configured to determine the breathing pattern by evaluating the detected heartbeats. The control unit is further configured to analyze the breathing pattern in order to determine the current diaphragm state. For example, in case the breathing pattern reaches a climax, the diaphragm state can be determined as being inhaled. In an analog way, a valley value of the breathing pattern can correspond to an exhaled diaphragm state.

According to a further embodiment, the control unit is configured to determine variations concerning a heart rate and/or variations concerning amplitudes of the heartbeats, wherein the heart rate may be derived from or may correspond to the reciprocal value of time intervals between subsequent heartbeats, and wherein the breathing pattern is determined based on said variations.

The control unit is configured to determine time intervals between subsequent heartbeats. Using that, the heart rate is the reciprocal value of the time intervals between subsequent heartbeats. The control unit is configured to determine variations between said heart rate. For example, it can be determined whether the heart rate is increasing or decreasing over time. In addition, the control unit is configured to determine the amplitudes of the heartbeats. The amplitude corresponds to the strength of the corresponding heart muscle contraction of a heartbeat. Using that, the control unit is configured to determine variations of the amplitudes. For example, it can be determined whether the amplitudes are increasing or decreasing over time. The control unit can be configured to determine the breathing pattern of the patient according to one of said variations or according to both variations. For example, an increasing amplitude can correspond to an increase within the breathing pattern.

According to a further embodiment, the at least one sensor is integrated within the medical imaging unit.

The at least one sensor can be integrated within the gantry of the medical imaging unit and/or be attached to the bed of the medical imaging unit. In particular, the at least one sensor is integrated in such way that a movement of the bed, for example relative to the gantry, does not interfere with the at least one sensor. This way, the at least one sensor does not influence the acquiring of the medical image in a disadvantageous manner. In addition, the at least one sensor is not exposed to the patient or the technician, thus significantly reducing the risk of accidentally detaching and/or damaging the at least one sensor.

According to a further embodiment, the at least one sensor is installed above and facing the patient and/or the at least one sensor is hooked up with the patient. In particular, the at least one sensor is hooked up with a leg, an arm, and/or the chest of the patient.

The term “hooked up” in this context particularly means that the at least one sensor is attached to the skin of the patient. For example, the at least one sensor can be temporarily glued to the leg, the arm, and/or the chest of the patient.

According to a further embodiment, the diaphragm state of the patient is stored with the medical image.

The diaphragm state can be stored as an additional tag with the medical image. The diaphragm state can be stored within a file containing the medical image. Hereby, the diaphragm state can be written as a label within the medical image and/or stored as an annex to the medical image.

According to a further embodiment, the control unit is configured for performing a reverse engineering process, wherein the reverse engineering process comprises the determination of the previous diaphragm state according to the previous medical image.

The reverse engineering process may be a model which is run by the control unit. In particular, the reverse engineering process can be designed as a deep learning algorithm. The control unit can be configured to use a deep learning algorithm for performing the reverse engineering process. The deep learning algorithm can be configured as a classifier in order to determine the previous diaphragm state shown corresponding to the previous medical image. During a learning phase of the deep learning algorithm, corresponding diaphragm states are assigned to a large number of medical images. That is used as a training data set of the deep learning algorithm. Hereby, the training data set is diverse and representative of a wide range of individuals ensuring that the trained deep learning algorithm is robust and capable of correctly identifying the diaphragm state of patients over a wide range of different age groups and of different gender. Especially, in case the previous diaphragm state of a previous medical image is not stored with the medical image, performing the reverse engineering process can still provide the information about the previous diaphragm state and thus ensure an acquiring of the current medical image with the same diaphragm state. Note, that other methods to perform the reverse engineering process are feasible as well.

According to a further embodiment, the control unit is configured to determine the previous diaphragm state according to the location and/or the size of at least one organ visible within the previous medical image.

Preferably, the deep learning algorithm is configured to identify a plurality of organs within the previous medical image and to determine the geometry and/or the locations of the organs. “Identifying” in this context particularly means that the deep learning algorithm can identify human organs within the previous medical image, for example liver, lung, intestine, spine, or the like. The geometry and/or the locations of the organs can be determined by edge detection. The geometry and/or the locations of the identified organs, in particular the lung, can be used as an indicator for the respective previous diaphragm state.

According to a second aspect, a method for using a system with a medical imaging unit for acquiring a medical image of a patient is suggested. The method comprises
acquiring sensor data of the patient by at least one sensor;
receiving the sensor data by an input unit; and
initiating the medical imaging unit based on the received sensor data by the control unit.

The system is embodied according to the first aspect or according to an embodiment of the first aspect.

According to an embodiment, the method further comprises determining a previous diaphragm state of the patient by the control unit; determining a current diaphragm state of the patient based on the received sensor data by the control unit; and comparing the current diaphragm state with the previous diaphragm state.

According to an embodiment, the determining of the previous diaphragm state of the patient is performed by storing the previous diaphragm state with the previous medical image and/or by a reverse engineering process analyzing the previous medical image.

According to a third aspect, a computer program product is proposed comprising instructions which, when the program is executed by a computer, cause the computer to carry out the above-mentioned method.

The computer program product, such as a computer program, may be embodied as a memory card, USB stick, CD-ROM, DVD or as a file which may be downloaded from a server in a network. For example, such a file may be provided by transferring the file comprising the computer program product from a wireless communication network.

The embodiments and features described with reference to the system of the first aspect apply mutatis mutandis to the method of the second aspect.

Further possible implementations or alternative solutions of embodiments of the present invention also encompass combinations—that are not explicitly mentioned herein—of features described above or be-low with regard to the embodiments. The person skilled in the art may also add individual or isolated aspects and features to the most basic form of the present invention.

### BRIEF DESCRIPTION OF THE DRAWINGS

Further embodiments, features and advantages of the present invention will become apparent from the subsequent description and dependent claims, taken in conjunction with the accompanying drawings, in which:

FIG. 1 shows a schematic perspective illustration of an embodiment of a system for acquiring medical images of a patient;

FIG. 2 shows a schematic illustration of an embodiment of sensor data acquired by an embodiment of an electrode of the system according to FIG. 1;

FIG. 3 shows a schematic illustration of an embodiment of a breathing pattern determined based on the sensor data according to FIG. 2;

FIG. 4 shows a schematic illustration of an embodiment of a temporal evolution of a current diaphragm state D determined based on the breathing pattern according to FIG. 3; and

FIG. 5 shows an embodiment of a method for using the system according to FIG. 1.

In the Figures, like reference numerals designate like or functionally equivalent elements, unless otherwise indicated.

### DETAILED DESCRIPTION

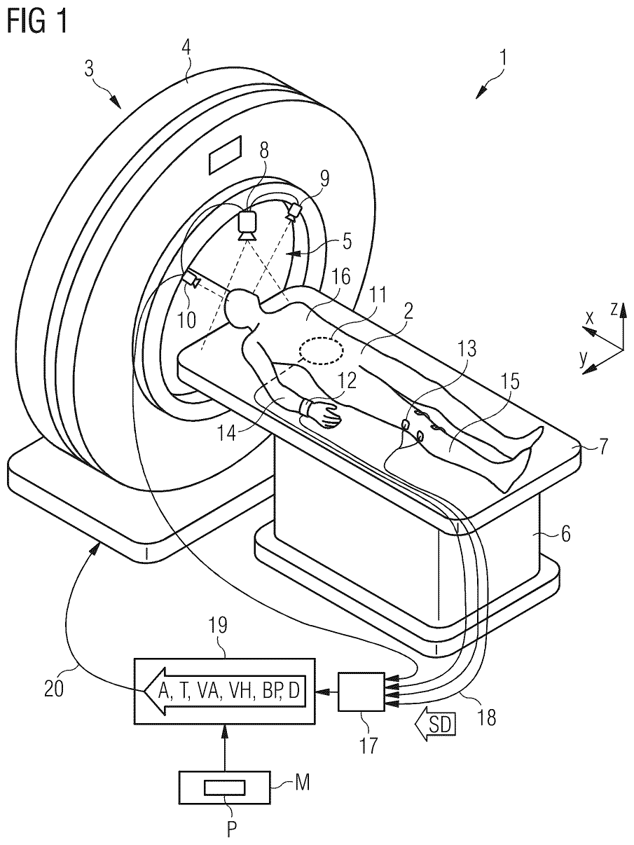

FIG. 1 shows a schematic perspective illustration of an embodiment of a system 1 for acquiring medical images of a patient 2. The system 1 comprises a medical imaging unit 3 with a gantry 4, whereby the gantry 4 contains an X-ray source and an X-ray detector (both not visible in FIG. 1). The gantry 4 surrounds a tube 5 in the shape of a narrow and long tunnel. The tube 5 extends along an axial direction x. The medical imaging unit 3 further comprises a table 6 onto which a bed 7 is attached. The bed 7 is attached to the table 6 such way that the bed 7 can be moved along the axial direction x. In particular, the bed 7 can be moved through the gantry 4.

The system 1 further comprises a plurality of sensors 8-13, in particular, it comprises a camera 8, two kinetic sensors 9, 10, a respiratory sensor 11, a blood pressure sensor 12, and an electrode 13. The camera 8 is integrated within the gantry 4 and on top of the bed 7 facing downwards. This way, the camera 8 is configured to capture the patient 2. The kinetic sensors 9, 10 are integrated within the gantry 4 as well at different locations inside the tube 5. This way, the kinetic sensors 9, 10 are configured to capture the patient 2 from different angles and thus enable capturing the patient 2 in a three-dimensional manner. The respiratory sensor 11 is integrated in the bed 7 underneath the patient 2. This way, the breathing status of the patient 2 can be detected by the respiratory sensor 11. The blood pressure sensor 12 is attached to an arm 14 of the patient 2 for detecting a blood pressure of the patient 2. The electrode 13 is attached to the arm 14, a leg 15, or a chest 16 of the patient 2 for detecting heartbeats H of the patient 2.

The sensors 8-13 are coupled with an input unit 17 via at least one sensor cable 18 of which only one is referred to with a reference sign in FIG. 1 Alternatively, or additionally, the sensors 8-13 can be coupled with the input unit 17 in a wireless manner. The input unit 17 converts sensor data SD of the sensor 8-13 into computer-readable digital data.

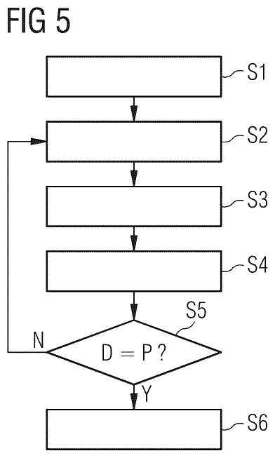

The system 1 further comprises a control unit 19 which is configured to derive a current diaphragm state D based on the sensor data SD. The control unit 19 will be described in detail below. Said current diaphragm state D corresponds to the current state of the lung of the patient 2. The current diaphragm state D can express one of the values “inhaled state” I, “exhaled state” E, and “in between state” B (see also FIG. 4). Note that there can exist other values to the current diaphragm state D as well.

The control unit 19 is configured to determine a previous diaphragm state P corresponding to a previous medical image M. The previous diaphragm state P can be stored with the previous medical image M. This way, the control unit 19 is configured to load the previous diaphragm state P from the previous medical image M. In case no information concerning the previous diaphragm state P is available, the control unit 19 is configured to perform a reverse engineering process. The reverse engineering process is a model which is run by the control unit 19. In particular, the reverse engineering process can be designed as a deep learning algorithm. Hereby, the control unit 19 is configured to identify a plurality of organs within the previous medical image M and to determine the geometry and/or the locations of the organs. “Identifying” in that context means that the deep learning algorithm can identify human organs within the previous medical image M, for example liver, lung, intestine, spine, or the like. The geometry and/or the locations of the organs is determined by edge detection. The geometry and/or the locations of the identified organs, in particular the lung, is used as an indicator for the respective previous diaphragm state P.

The control unit 19 is configured to compare the previous diaphragm state P with the current diaphragm state D. If they are not in agreement with one another, the control unit 19 is configured to further acquire sensor data SD and to determine the corresponding current diaphragm state D. If they are identical, the control unit 19 is configured to automatically initiate the medical imaging unit 3 to acquire a medical image of the patient 2, for example via an output cable 20 or via a wireless connection with the medical imaging unit 3. The control unit 19 is further configured to inform a technician (not visible in the figures) using the medical imaging unit 3 about the determined current diaphragm state D, possibly by an acoustic and/or a visual indication. The control unit 19 can be part of a computer program product for controlling the medical imaging unit 3.

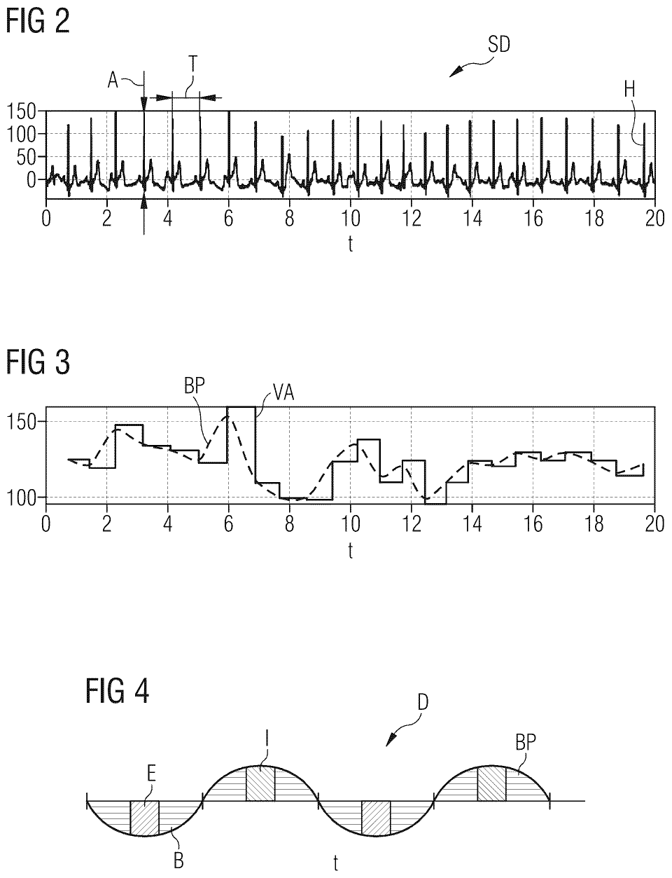

FIG. 2 shows a schematic illustration of an embodiment of sensor data SD acquired by the electrode 13. The sensor data SD comprise electrical signals produced by muscle contractions of the patient 2. In case the patient 2 is not moving, said muscle contractions represent the activity of the heart of the patient 2. Thus, the sensor data SD of the electrode 13 show heartbeats H of the patient 2, represented as peaks within the sensor data SD. Note, that in FIG. 2, only one peak is referred to with a reference number.

The control unit 19 is configured to determine time intervals T between subsequent heartbeats H as well as an amplitude A of the heartbeats H, of which each only one is assigned with a reference number in FIG. 2. The amplitude A corresponds to the strength of the corresponding heart muscle contraction. It can be seen that over time, the amplitude A shows variations. Using the time intervals T, a heart rate is derived from or may correspond to the reciprocal value of the time intervals T.

FIG. 3 shows a schematic illustration of an embodiment of a breathing pattern BP determined based on the sensor data SD as described above. The control unit 19 is configured to determine variations VH of the heart rate over time. Additionally, the control unit 19 is configured to determine variations VA of the amplitudes A over time. An increase of the amplitudes A during a time interval results in an upwards-directed step of the variations VA of the amplitudes A within that time interval. The control unit 19 is configured to determine the breathing pattern BP of the patient 2 according to the variations VA of the amplitudes A and/or according to the variations VH of the heart rate. Hereby, an increase regarding at least one of said variations VA, VH can correspond to an increase within the breathing pattern BP.

FIG. 4 shows a schematic illustration of an embodiment of a temporal evolution of the current diaphragm state D as determined based on the breathing pattern BP described above. The control unit 19 is configured to determine the current diaphragm state D based on the breathing pattern BP. For example, in case the breathing pattern BP reaches a climax, the current diaphragm state D can be determined as “inhaled state” I. In an analog way, a valley value of the breathing pattern BP corresponds to the diaphragm state D being “exhaled state” E.

FIG. 5 shows an embodiment of a method for using the system 1 as described above. In a first step S1, the previous diaphragm state P is determined by the control unit 19 based on a reverse engineering process of the previous medical image M or by reading the previous diaphragm state P stored with the previous medical image M. In a second step S2, the sensor data SD of the patient 2 are acquired by the at least one sensor 8-13. In a third step S3, the sensor data SD are received by an input unit 17. In a fourth step S4, the current diaphragm state D is determined by the control unit 19 based on the received sensor data SD. In a fifth step S5, the determined current diaphragm state D and the determined previous diaphragm state P are compared with one another. In case they match with one another, the medical imaging unit 3 is initiated to acquire the medical image by the control unit 19 in a sixth step S6. In case the current diaphragm state D and the previous diaphragm state P do not match, the second step S2 is repeated.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A system for acquiring a medical image of a patient, the system comprising:
a medical imaging unit configured to acquire the medical image;
at least one sensor configured to acquire sensor data of the patient;
an input unit configured to receive the sensor data; and
a control unit configured to initiate acquisition of the medical image based on the sensor data.

2. The system according to claim 1, wherein the control unit is configured to initiate the acquisition of the medical image in case a current diaphragm state of the patient corresponds to a previous diaphragm state of the patient.

3. The system according to claim 2, wherein the at least one sensor is an electrode configured to detect heartbeats of the patient.

4. The system according to claim 3, wherein the control unit is configured to determine the current diaphragm state of the patient based on the heartbeats.

5. The system according to claim 4, wherein
the control unit is configured to determine a breathing pattern of the patient based on the heartbeats, and
the control unit is configured to determine the current diaphragm state based on the breathing pattern.

6. The system according to claim 5, wherein
the control unit is configured to determine at least one of variations concerning a heart rate or variations concerning amplitudes of the heartbeats,
the heart rate is derived from, or corresponds to, a reciprocal value of time intervals between subsequent heartbeats, and
the breathing pattern is determined based on said variations.

7. The system according to claim 1, wherein the at least one sensor is integrated within the medical imaging unit.

8. The system according to claim 7, wherein at least one of
the at least one sensor is installed above and facing the patient, or
the at least one sensor is hooked up to the patient.

9. The system according to claim 2, wherein the previous diaphragm state of the patient is stored with a previous medical image.

10. The system according to claim 2, wherein the control unit is configured to perform a reverse engineering process, wherein the reverse engineering process includes a determination of the previous diaphragm state according to a previous medical image.

11. The system according to claim 10, wherein the control unit is configured to determine the previous diaphragm state according to at least one of a location or a size of at least one organ visible within the previous medical image.

12. A method for using a system with a medical imaging unit for acquiring a medical image of a patient, the method comprising:
acquiring, by at least one sensor, sensor data of the patient;
receiving the sensor data by an input unit; and
initiating, by a control unit, the acquiring of the medical image based on the sensor data.

13. The method according to claim 12, further comprising:
determining, by the control unit, a previous diaphragm state of the patient;
determining, by the control unit, a current diaphragm state of the patient based on the sensor data; and
comparing the current diaphragm state with the previous diaphragm state.

14. The method according to claim 13, wherein the determining of the previous diaphragm state of the patient is performed by at least one of storing the previous diaphragm state with a previous medical image or a reverse engineering process analyzing the previous medical image.

15. A non-transitory computer-readable storage medium storing computer-executable instructions that, when executed by a computer, cause the computer to carry out the method of claim 12.

16. The system according to claim 8, wherein the at least one sensor is hooked up to at least one of a leg, an arm, or a chest of the patient.

17. The system according to claim 3, wherein the control unit is configured to perform a reverse engineering process, wherein the reverse engineering process includes a determination of the previous diaphragm state according to a previous medical image.

18. The system according to claim 17, wherein the control unit is configured to determine the previous diaphragm state according to at least one of a location or a size of at least one organ visible within the previous medical image.

19. The system according to claim 4, wherein the control unit is configured to perform a reverse engineering process, wherein the reverse engineering process includes a determination of the previous diaphragm state according to a previous medical image.

20. The system according to claim 5, wherein the control unit is configured to perform a reverse engineering process, wherein the reverse engineering process includes a determination of the previous diaphragm state according to a previous medical image.
